(19) United States
(12) Patent Application Publication
(10) Pub. No.: US 2010/0183694 A1
(43) Pub. Date: Jul. 22, 2010

(54) URINAL FILTER

(76) Inventors: Robert B. Burke, Ridgewood, NJ (US); John B. Earl, Ridgewood, NJ (US)

Correspondence Address: REISING ETHINGTON P.C., P O BOX 4390, TROY, MI, 48099-4390, US

(21) Appl. No.: 12/357,051

(22) Filed: Jan. 21, 2009

(51) Int. Cl. A01N 25/34; A01P 15/00; A61L 9/012

(52) U.S. Cl. 424/411; 424/400; 424/76.7

(57) ABSTRACT

One exemplary embodiment includes a urinal filter including a foam.

### TECHNICAL FIELD

The field to which the disclosure generally relates to includes urinary filters.

### BACKGROUND

Men's urinals typically include a filter over the drain opening thereof. Such filters include a relatively hard plastic cage which houses a deodorizing puck or block. Although the materials used for the puck or block may vary, they commonly include pure paradichlorobencene.

### SUMMARY OF EXEMPLARY EMBODIMENTS OF THE INVENTION

One exemplary embodiment includes a urinal filter comprising a foam.

Other exemplary embodiments of the invention will become apparent from the detailed description provided hereinafter. It should be understood that the detailed description and specific examples, while disclosing exemplary embodiments of the invention, are intended for purposes of illustration only and are not intended to limit the scope of the invention.

### BRIEF DESCRIPTION OF THE DRAWINGS

Exemplary embodiments of the invention will become more fully understood from the detailed description and the accompanying drawings, wherein:

FIG. 1 is a perspective view of a urinal including a urinal filter according to one exemplary embodiment.

FIG. 2 is a plan view of a urinal filter according to one exemplary embodiment.

FIG. 3 is a side view of a urinal filter according to one exemplary embodiment.

FIG. 4 is a plan view of a urinal filter including indicia thereon according to one exemplary embodiment.

### DETAILED DESCRIPTION OF EXEMPLARY EMBODIMENTS

The following description of the embodiment(s) is merely exemplary (illustrative) in nature and is in no way intended to limit the invention, its application, or uses.

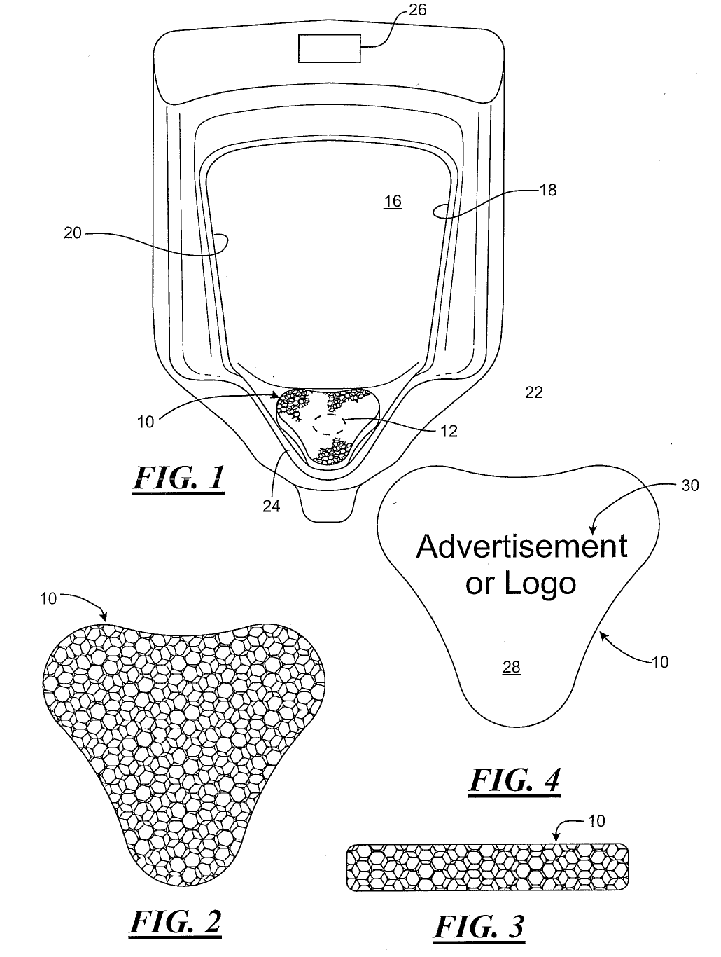

Referring to FIG. 1, in one exemplary embodiment a filter 10 including a foam mat may be placed over a drain 12 of a men's urinal 14 to prevent debris, such as cigarettes and paper, from going down the urinal drain 12 and plugging the same. The urinal may be of any of a variety known to those skilled in the art. In the embodiment illustrated by FIG. 1 the urinal 14 may include a back wall 16, opposite side walls 18, 20, a trough 22 defined at least in part by a floor 24 having an opening or drain 12 formed therein. A water supply 26 may be coupled to the urinal to rinse or flush waste therefrom. The foam filter 10 may include a deodorizer or a order masking agent such as a fragrance, and a biocide such as a antibacterial agent or antifungus agent. Any of a variety of foams may be utilized. In one embodiment, the foam is a reticulated foam. The reticulated foam may include a plurality of open or closed cells. The foam may be constructed and arranged to prevent or extremely limit the amount of splash that may occur when a user urinates into the urinal.

The foam may be made by a variety of methods and material. For example, the foam may be made by the mixture of a polyol, an isocyanate and a blowing agent as necessary. The deodorizer or biocide may be added to (pre-mixed with) the polyol, the isocyanate, or the blowing agent or mixed in with the three just prior to depositing the foam mixture on a surface.

Alternatively, the deodorizer may be deposited on the foam filter 10 and kept in a wet state by inserting the mat with the liquid fragrance thereon any plastic bag and sealing the same. The wet mat may be removed from the plastic bag and deposited in the urinal.

It has be found that when a deodorizer is deposited on the foam with the inclusion of a binder, the binder makes the foam more rigid and results in greater splash than foams wherein the deodorizer is mixed with the polyol and isocyanate or wherein the deodorizer is deposited on the foam without a binder and the foam is kept in a wet state until being deposited in the urinal. A suitable biocide is available from Rohm and Haas under the trade name Rocima 200. In one embodiment, the urinal filter 10 may be a flat foam mat having a thickness of about ⅜ inches and formed in a generally triangular shape.

Referring to FIG. 4, if desired, an upper surface 28 of the foam filter 10 which may be deposited in the urinal may be a mat including an upper surface having an advertisement, logo, writing, indicia or similar information communication material 30 printed, molded or machined on or therein. For example, the upper surface of the foam may be stamped with an ink to deposit a logo associated with a sports team or product.

Suitable polyols include, but are not limited to, those as are conventionally used in the preparation of rigid polyurethane foam and which typically have an average hydroxyl equivalent weight of from about 50 to about 700, preferably from about 70 to about 500, more preferably from about 70 to about 300. Additionally, such polyols may generally contain from about 2 to about 8, preferably from about 3 to about 8, and more preferably from about 3 to about 6 hydroxyl groups per molecule. Examples of suitable polyols are polyether polyols as described more fully in U.S. Pat. No. 4,394,491. Exemplary of such polyether polyols include those commercially available under the trademark, VORANOL and include VORANOL 202, VORANOL 360, VORANOL 370, VORANOL 446, VORANOL 490, VORANOL 575, VORANOL 640, VORANOL 800 all sold by The Dow Chemical Company. Other polyols include alkylene oxide derivatives of Mannich condensate as taught in, for example, U.S. Pat. Nos. 3,297,597; 4,137,265 and 4,383,102, and amino-alkylpiperazine-initiated polyether polyols as described in U.S. Pat. Nos. 4,704,410 and 4,704,411. A group of polyester polyols are also.

Suitable isocyanates include, but are not limited to, aromatic, aliphatic and cycloaliphatic polyisocyanates and combinations thereof. A crude polyisocyanate may also be used in the practice of this invention, such as the crude toluene diisocyanate obtained by the phosgenation of a mixture of toluene diamines or the crude diphenylmethane diisocyanate obtained by the phosgenation of crude methylene diphenylamine. Suitable polyisocyanates are also disclosed in U.S. Pat. No. 3,215,652. Examples include methylene-bridged polyphenyl polyisocyanates and mixtures thereof with crude diphenylmethane diisocyanate, due to their ability to cross-link the polyurethane. The amount of polyisocyanate present when preparing the foam may vary. For example 0.6 to about 3.0 isocyanate groups per isocyanate reactive atom present in the polyol(s) and any water as may be present. The amount of isocyanate may also vary, for example, from about 0.7, more preferably from about 0.8, and preferably up to about 2, more preferably up to about 1.6 isocyanate groups per isocyanate reactive atom.

A blowing agent may be present in an amount to provide the cellular polymer with a desired overall density, which may be, for example, from about 10 to about 150, preferably from about 10 to about 100, and more preferably from about 15 to about 80 kg/m³. Suitable blowing agents include, but are not limited to, alkanes such as pentane, hexane or a halogen-containing substances such as (per)fluorocarbons and the hydrogen-containing chlorofluorocarbon compounds exemplary of which include Refrigerant 21, Refrigerant 22, Refrigerant 123, Refrigerant 123a, Refrigerant 124, Refrigerant 124a, Refrigerant 133 (all isomers), Refrigerant 134, Refrigerant 141b, Refrigerant 142, Refrigerant 151. Among these, Refrigerant 123 (all isomers), Refrigerant 141b and Refrigerant 142 (all isomers) are most preferred, as these are commercially available in addition to being recognized as having low ozone depletion potentials. Other blowing agents also include the amine/carbon dioxide complexes such as taught in U.S. Pat. Nos. 4,735,970 and 4,500,656.

Suitable deodorizers are available from Andrea Aromatics including, but not limited to, Wintergreen, Spearmint, Berry, Washed Lines and Aloe fragrances.

Examples of suitable biocides include, but are not limited to, monopersulfate compounds, copper sulfate, silver nitrate, and mixtures thereof. Other suitable biocides include 1,4-dichlorobenzene, glueraldehyde or formaldehyde. However, when an advertisement or logo is provided on the foam, oxidizing agents could damage the advertisement or logo. Examples of non-oxidizing biocides that may be used with a foam having an advertisement or logo may include, but is not limited to, quaternary surfactants.

The above description of embodiments of the invention is merely exemplary in nature and, thus, variations thereof are not to be regarded as a departure from the spirit and scope of the invention.

What is claimed is:

1. A urinal filter comprising:
a foam including at least one of a deodorizer or a biocide.

2. A urinal filter as set forth in claim 1 wherein the foam is one of opened cell reticulated foam or closed cell reticulated foam.

3. A urinal filter as set forth in claim 1 wherein the foam includes the deodorizer.

4. A urinal filter as set forth in claim 1 wherein the foam includes the biocide.

5. A urinal filter as set forth in claim 1 wherein the foam includes an upper surface having an advertisement, logo, writing, indicia or similar information communication material printed, molded or machined on or therein.

6. A method of filtering material in a urinal including a drain comprising:
placing a filter comprising a foam over the drain.

7. A method as set forth in claim 6 wherein the foam comprising at least one of a deodorizer or a biocide.

8. A method as set forth in claim 6 wherein the foam is one of opened cell reticulated foam or closed cell reticulated foam.

9. A method as set forth in claim 6 wherein the foam includes the deodorizer.

10. A method as set forth in claim 6 wherein the foam includes the biocide.

11. A method as set forth in claim 6 wherein the foam includes an upper surface having an advertisement, logo, writing, indicia or similar information communication material printed, molded or machined on or therein.

12. A method comprising:
mixing a polyol, an isocyanate and at least one of a deodorizer or a biocide so that the polyol and isocyanate form a foam including the deodorizer or biocide.

13. A method as set forth in claim 12 wherein the deodorizer or biocide is mixed first with one of the polyol or isocyante prior to mixing the polyol and isocyanante.

14. A method as set forth in claim 12 further comprising mixing a blowing agent with the polyol and isocyanate.

15. A method as set forth in claim 14 wherein the deodorizer or biocide is mixed first with one of the polyol, isocyante or blowing agent prior to mixing the polyol and isocyanante.

16. A method as set forth in claim 12 wherein the foam includes the deodorizer.

17. A method as set forth in claim 12 wherein the foam includes the biocide.

18. A method as set forth in claim 12 further comprising placing the foam over a drain of a urinal.
